(19) United States
(12) Patent Application Publication
(10) Pub. No.: US 2021/0177520 A1
(43) Pub. Date: Jun. 17, 2021

(54) CALIBRATION DEVICE FOR SURGICAL INSTRUMENTS

(71) Applicant: METAL INDUSTRIES RESEARCH & DEVELOPMENT CENTRE, Kaohsiung City (TW)

(72) Inventors: Ming-Hui Chen, Kaohsiung City (TW); Kai-Shi Luo, Tainan City (TW)

(21) Appl. No.: 16/713,114

(22) Filed: Dec. 13, 2019

(51) Int. Cl. A61B 34/20; A61B 34/00; G01B 11/03

(52) U.S. Cl. CPC A61B 34/20; A61B 2034/207; G01B 11/03; A61B 34/70

(57) ABSTRACT

A calibration device for surgical instruments includes a base, an instrument holder and at least one reference frame marker, the instrument holder is pivotally mounted on the base, and the reference frame marker is mounted on the base. The surgical instrument can be inserted into an insertion hole of the instrument holder to rotate or swing selectively on the base such that a signal processor of a surgical navigation system can receive coordinate signals from the reference frame marker and at least one coordinate marker mounted on the surgical instrument to calibrate the surgical instrument.

### FIELD OF THE INVENTION

This invention generally relates to a calibration device, and more particularly to a calibration device used to calibrate coordinates of surgical instruments.

### BACKGROUND OF THE INVENTION

Surgical navigation system is a necessary auxiliary system for minimally invasive surgeries (brain surgery, vertebrae surgery and abdominal puncture). Before surgery, positioning of navigation system and surgical instruments, such as surgical probe and bone access needle, is required for obtaining surgical instrument coordinate in the surgical navigation system.

China Patent Application No. 201410396220.0 (Publication No. CN 104146773 A) disclosed a calibration block used for calibrating surgical instruments. However, calibration error may be caused due to the calibration block is unavailable for calibrating six degrees of freedom and straightness of surgical instruments. Accuracy and precision in conventional surgical navigation system have to be improved.

### SUMMARY

One object of the present invention is to utilize an instrument holder pivotally mounted on a base to allow a surgical instrument inserted into the instrument holder to selectively rotate or swing for calibrating six degrees of freedom and straightness of the surgical instrument.

A calibration device of the present invention includes a base, an instrument holder and at least one reference frame marker, the instrument holder is pivotally mounted on the base and includes at least one insertion hole, the at least one insertion hole is provided for insertion of a surgical instrument such that the surgical instrument is able to be rotated or swung selectively on the base, the reference frame marker is mounted on the base.

Because of the instrument holder pivotally mounted on the base, the surgical instrument inserted into the instrument holder can be rotated or swung selectively on the base, consequently, a signal processor can receive coordinate signals from the reference frame marker mounted on the base and at least one coordinate marker mounted on the surgical instrument for calibrating the surgical instrument.

### DESCRIPTION OF THE DRAWINGS

FIG. 1 is a perspective assembly diagram illustrating a calibration device in accordance with a first embodiment of the present invention and a surgical instrument.

FIG. 2 is a perspective explored diagram illustrating the calibration device in accordance with the first embodiment of the present invention.

FIG. 3 is a cross-section view diagram illustrating the calibration device in accordance with the first embodiment of the present invention.

FIG. 4 is a perspective assembly diagram illustrating insertion of a surgical instrument with the calibration device in accordance with the first embodiment of the present invention.

FIG. 5 is a perspective assembly diagram illustrating a calibration device in accordance with a second embodiment of the present invention.

FIG. 6 is a partial perspective assembly diagram illustrating the calibration device in accordance with the second embodiment of the present invention.

FIG. 7 is a perspective explored diagram illustrating the calibration device in accordance with the second embodiment of the present invention.

FIG. 8 is a cross-section view diagram illustrating the calibration device in accordance with the second embodiment of the present invention.

FIG. 9 is a cross-section view diagram illustrating the calibration device in accordance with the second embodiment of the present invention.

FIG. 10 is a cross-section view diagram illustrating the calibration device in accordance with the second embodiment of the present invention.

FIG. 11 is a perspective explored diagram illustrating a calibration device in accordance with a third embodiment of the present invention.

FIG. 12 is a cross-section view diagram illustrating the calibration device in accordance with the third embodiment of the present invention.

FIG. 13 is a perspective assembly diagram illustrating a calibration device in accordance with a fourth embodiment of the present invention.

FIG. 14 is a partial perspective assembly diagram illustrating the calibration device in accordance with the fourth embodiment of the present invention.

FIG. 15 is a perspective explored diagram illustrating the calibration device in accordance with the fourth embodiment of the present invention.

FIG. 16 is a cross-section view diagram illustrating the calibration device in accordance with the fourth embodiment of the present invention.

FIG. 17 is a cross-section view diagram illustrating the calibration device in accordance with the fourth embodiment of the present invention.

FIG. 18 is a cross-section view diagram illustrating the calibration device in accordance with the fourth embodiment of the present invention.

FIG. 19 is a perspective explored diagram illustrating a calibration device in accordance with a fifth embodiment of the present invention.

FIG. 20 is a cross-section view diagram illustrating the calibration device in accordance with the fifth embodiment of the present invention.

FIG. 21 is a perspective explored diagram illustrating a calibration device in accordance with a sixth embodiment of the present invention.

FIG. 22 is a cross-section view diagram illustration the calibration device in accordance with the sixth embodiment of the present invention.

FIG. 23 is a cross-section view diagram illustrating a calibration device in accordance with a seventh embodiment of the present invention.

### DETAILED DESCRIPTION OF THE INVENTION

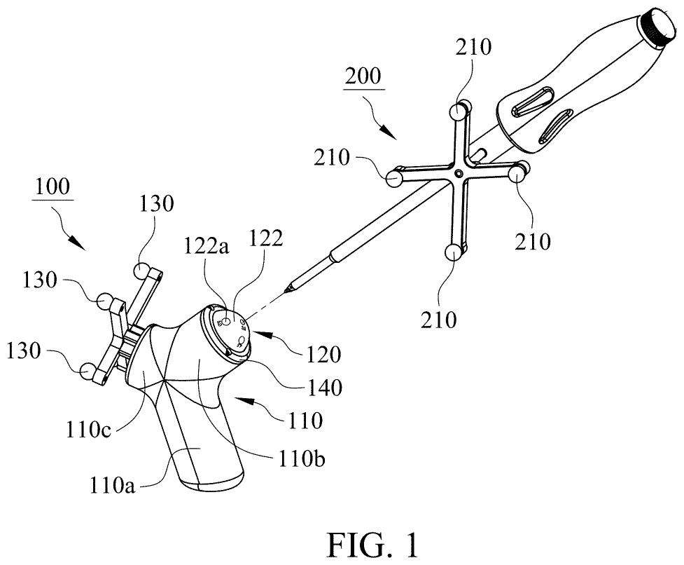

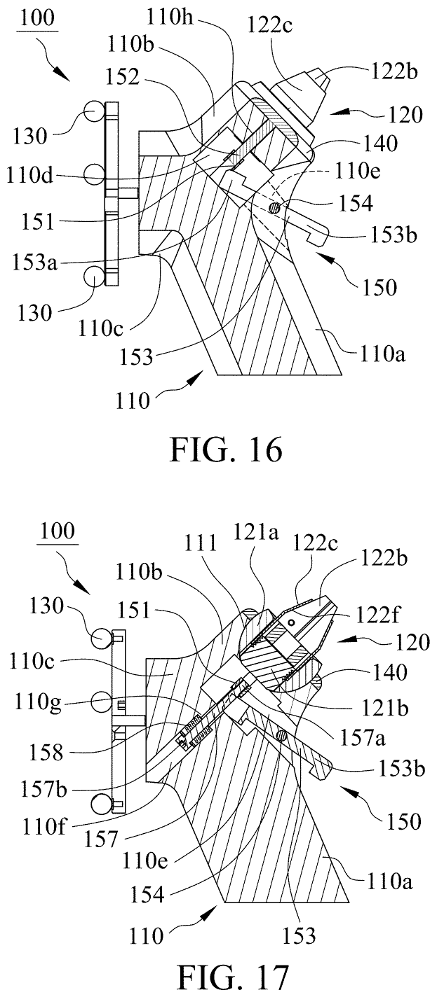

With reference to FIGS. 1 to 23, a calibration device 100 is provided for coordinate calibration of a surgical instrument 200, such as surgical probe or bone access needle. The calibration device 100 includes a base 110, an instrument holder 120 and at least one reference frame marker 130. The reference frame marker 130 is mounted on the base 110, and the instrument holder 120 is pivotally mounted on the base 110, as a result, the instrument holder 120 can rotate or swing selectively relative to the base 110. There is at least one insertion hole 122a formed on the instrument holder 120, and the surgical instrument 200 is able to be inserted into the insertion hole 122a and able to be rotated or swung selectively with the instrument holder 120 on the base 110. At least one coordinate marker 210 is mounted on the surgical instrument 200 to be moved with the surgical instrument 200. The reference frame marker 130 and the coordinate marker 210 are, but not limited to, image sensors or radio frequency components.

The calibration device 100 of a first embodiment of the present invention is shown in FIGS. 1 to 4. The instrument holder 120 is pivotally mounted in a first accommodation groove 111 of the base 110, and in the first embodiment, the instrument holder 120 includes a pivot portion 121 and an insertion portion 122. The pivot portion 121 is pivotally located in the first accommodation groove 111 so the instrument holder 120 is rotatable or swingable, and the pivot portion 121 and the insertion portion 122 are connected to one another and movable together. The insertion hole 122a is located on the insertion portion 122 and is revealed by the first accommodation grove 111.

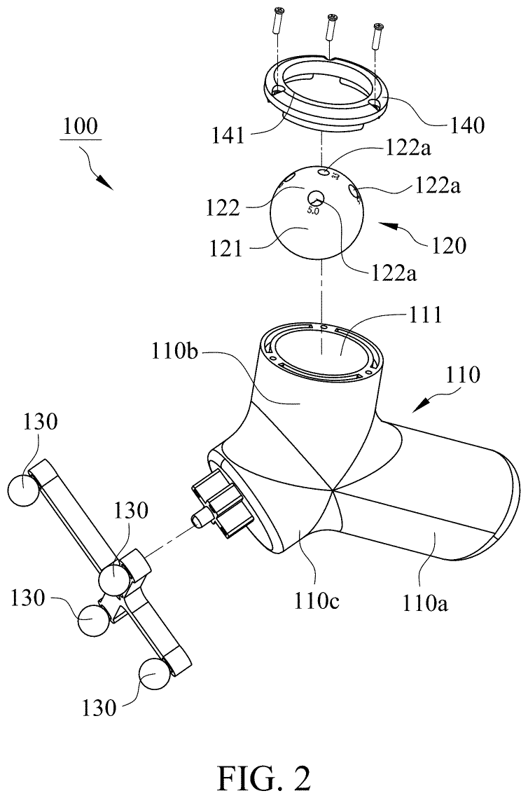

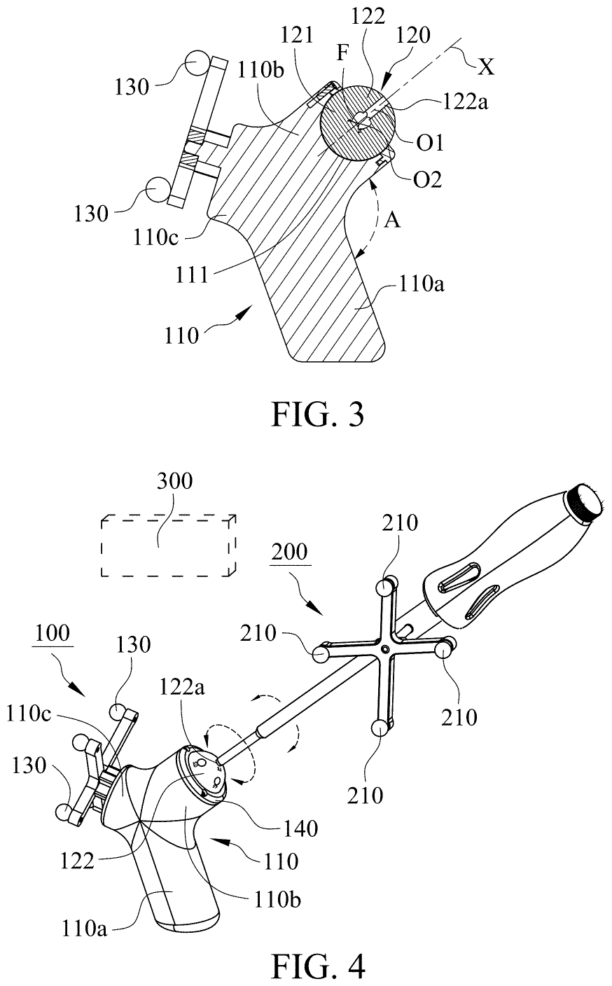

With reference to FIGS. 1 to 4, the base 110 of the first embodiment includes a grip portion 110a, a pivot portion 110b and a marker mounting portion 110c. As shown in FIGS. 2 and 3, the grip portion 110a is provided for user to grip, the pivot portion 110b is coupled to the grip portion 110a, and an included angle A exists between the pivot portion 110b and the grip portion 110a, the first accommodation groove 111 is recessed on the pivot portion 110b, the reference frame marker 130 is mounted on the marker mounting portion 110c. With reference to FIG. 3, in the first embodiment, the instrument holder 120 is a spheroid, an axis X passes through a center O1 of the insertion hole 122a and a center of gravity O2 of the instrument holder 120, and the center of gravity O2 is located on a hole bottom surface F of the insertion hole 122a. Preferably, there are multiple insertion holes 122a on the insertion portion 122, each of the insertion holes 122a has a diameter different to one another for insertion of surgical instruments 200 having different outer diameters. If the insertion portion 122 has multiple insertion holes 122a, there are multiple axes X which each passes through center O1 of each of the insertion holes 122a and the center of gravity O2 of the instrument holder 120, further, the center of gravity O2 is located on hole bottom surface F of each of the insertion holes 122a such that terminals of the surgical instruments 200 inserted into different insertion holes 122a all can align the center of gravity O2.

With reference to FIG. 4, after inserting the surgical instrument 200 into the insertion hole 122a, a signal processor 300 is provided to receive a first coordinate signal of the reference frame marker 130 mounted on the base 110 and a second coordinate signal of the coordinate marker 210 mounted on the surgical instrument 200 for calibrating a straightness of the surgical instrument 200. And then when the surgical instrument 200 is moved (swung or rotated), the signal processor 300 is provided to receive the second coordinate signal after moving to calibrate the degrees of freedom of the surgical instrument 200 in 3D space.

With reference to FIG. 1 to 3, the calibration device 100 preferably further includes a restriction lid 140 which has an exposure hole 141 communicating with the first accommodation groove 111. The restriction lid 140 is configured to cover the pivot portion 121 to cage the pivot portion 121 between the first accommodation groove 111 and the restriction lid 140, and the insertion hole 122a is exposed by the exposure hole 141. In the first embodiment, the restriction lid 140 is provided to contact the pivot portion 121 to prevent improper rotation of the instrument holder 120.

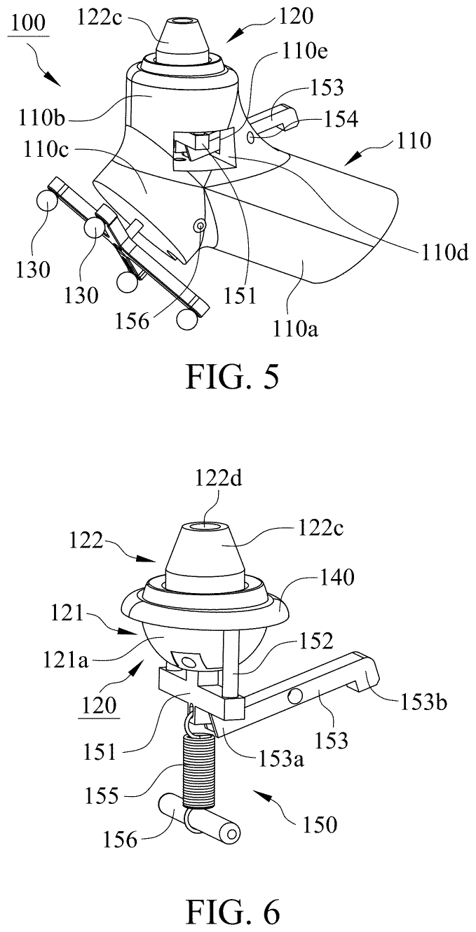

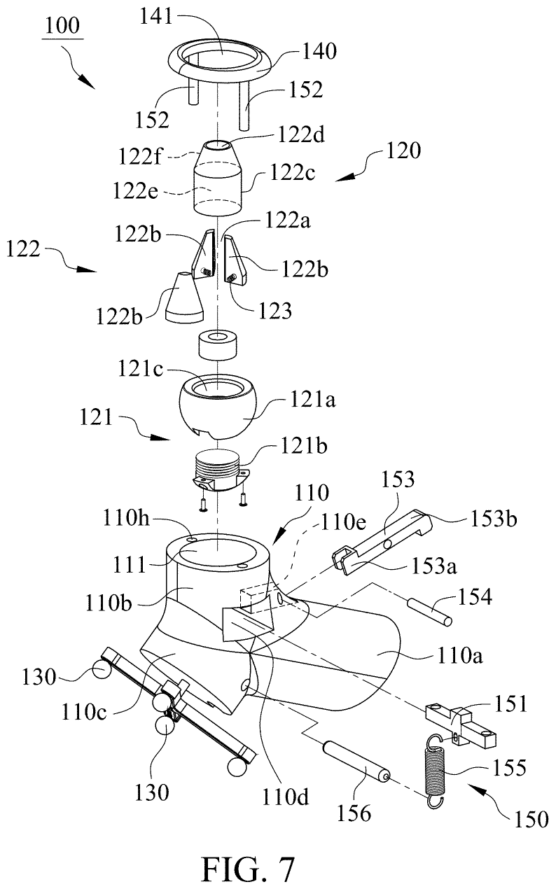

FIGS. 5 to 10 are diagrams illustrating a calibration device 100 of a second embodiment of the present invention. The insertion portion 122 and the pivot portion 121 of the second embodiment are different to those of the first embodiment. With reference to FIGS. 6 to 9, in the second embodiment, the insertion portion 122 includes a plurality of grippers 122b and a sleeve 122c, and the pivot portion 121 includes a body 121a and a screw post 121b. The sleeve 122c includes an opening 122d, an accommodation space 122e and a conical inner wall 122f, the opening 122d communicates with the accommodation space 122e, and the conical inner wall 122f surrounds around the accommodation space 122e. The body 121a has a second accommodation groove 121c, and the screw post 121b is located in the second accommodation groove 121c. The sleeve 122c is screwed with the screw post 121b, the grippers 122b are placed in the accommodation space 122e and surround around the insertion hole 122a, and the conical inner wall 122f of the sleeve 122c contacts the grippers 122b. Preferably, each of the grippers 122b has an elastic element 123. With reference to FIGS. 7 and 9, the sleeve 122c can be rotated to change the screwing position relative to the screw post 121b and further change the diameter of the insertion hole 122a. Consequently, the insertion hole 122a with adjustable diameter is suitable for different surgical instruments 200. With reference to FIGS. 7 and 9, in order to enlarge the diameter of the insertion hole 122a, the sleeve 122c screwed with the screw post 121b can be rotated up to allow the elastic elements 123 to push the grippers 122b to touch the conical inner wall 122f. In contrast, the diameter of the insertion hole 122a can be reduced by rotating the sleeve 122c screwed with the screw post 121b down to allow the conical inner wall 122f to contact the grippers 122b. In the second embodiment, the diameter of the insertion hole 122a can be adjusted by rotating the sleeve 122c up or down to change the screwing position of the sleeve 122c on the screw post 121b, as a result, the grippers 122b can be used to grip different surgical instruments 200.

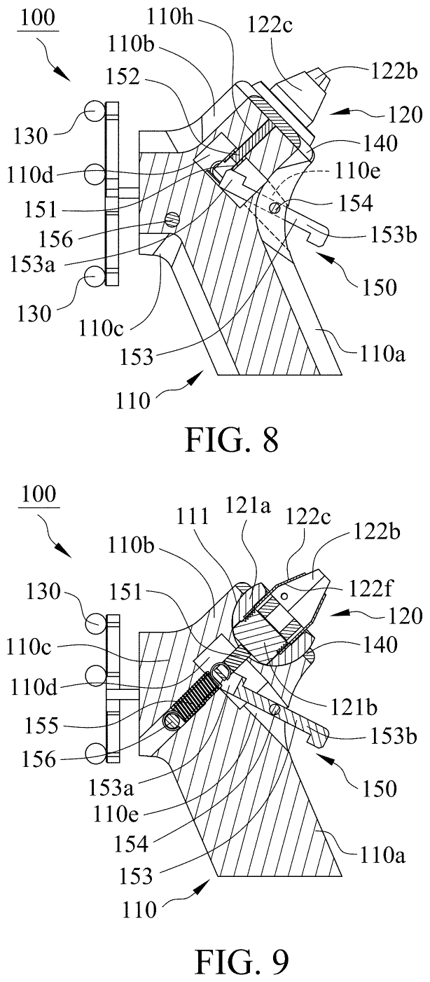

With reference to FIGS. 5 to 10, the calibration device 100 further includes a release mechanism 150 in the second embodiment. The release mechanism 150 is provided to allow the restriction lid 140 not to touch the instrument holder 120 such that the instrument holder 120 can be rotated or swung freely. With reference to FIGS. 6 and 7, the release mechanism 150 includes a beam 151, at least one transmission rod 152 and a lever 153. With reference to FIGS. 5, 7 and 9, the base 110 has a first space 110d and a second space 110e which communicate with each other, the beam 151 is set in the first space 110d, and the lever 153 is set in the second space 110e. With reference to FIGS. 7 and 8, the transmission rod 152 is movably located in a through hole 110h of the pivot portion 110b, one end of the transmission rod 152 is connected to the beam 151 and the other end of the transmission rod 152 is connected to the restriction lid 140. The lever 153 is pivotally mounted in the second space 110e by a pivot 154 and includes a pushing end 153a and a pressed end 153b, the pushing end 153a is located in the first space 110d and under the beam 151, the pressed end 153b protrudes from the second space 110e.

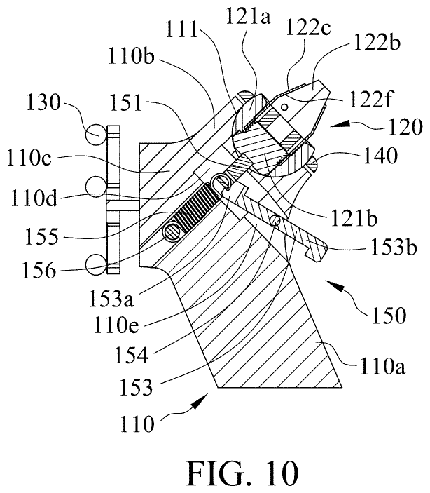

With reference to FIG. 10, while the pressed end 153b is pressed down, the pushing end 153a is provided to push the beam 151 to allow the beam 151, the transmission rod 152 and the restriction lid 140 to move toward a first direction together and allow the restriction lid 140 to release the pivot portion 121 such that the instrument holder 120 is free to rotate or swing in the absence of the restriction lid 140.

With reference to FIGS. 5 to 10, preferably, the release mechanism 150 further includes at least one elastic element 155, and both ends of the elastic element 155 are fixed on the base 110 and the beam 151, respectively. As shown in FIGS. 9 and 10, when no external force acts on the pressed end 153b, the elastic element 155 is configured to pull the beam 151 such that the beam 151, the transmission rod 152 and the restriction lid 140 are moved toward a second direction opposite to the first direction to allow the restriction lid 140 to contact the pivot portion 121. In the second embodiment, the release mechanism 150 further includes a positioning element 156 which is mounted in the base 110 to fix the elastic element 155 in the base 110.

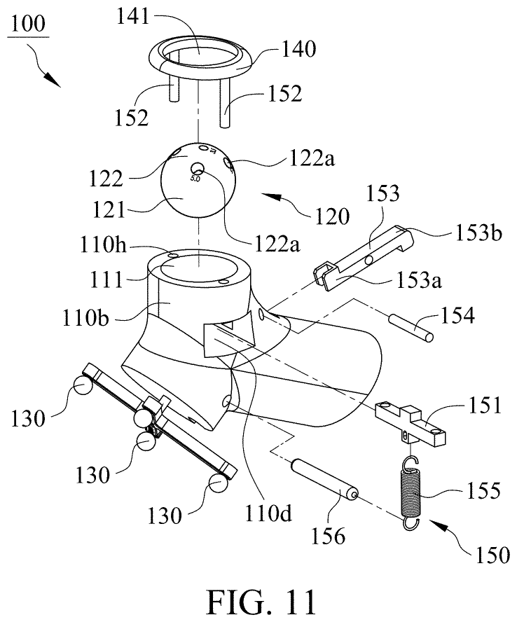

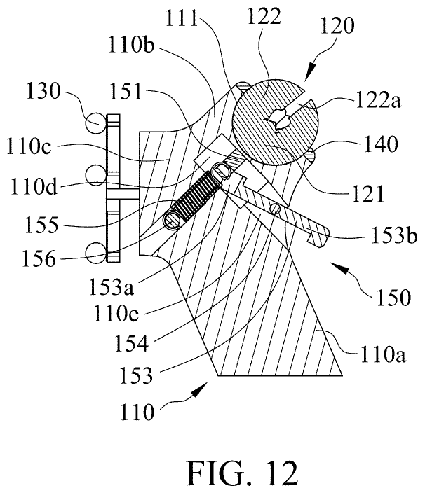

A calibration device 100 of a third embodiment of the present invention is shown in FIGS. 11 and 12. The instrument holder 120 is a spheroid in the third embodiment, different to the instrument holder 120 of the second embodiment. In the third embodiment, the release mechanism 150 is also provided to allow the restriction lid 140 to block the pivot portion 121 or allow the restriction lid 140 to unblock the pivot portion 121 by pressing the lever 153.

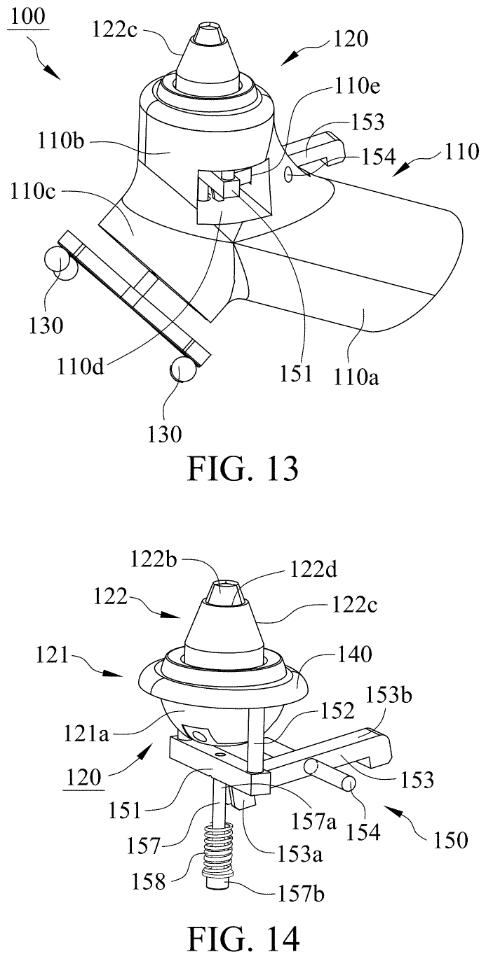

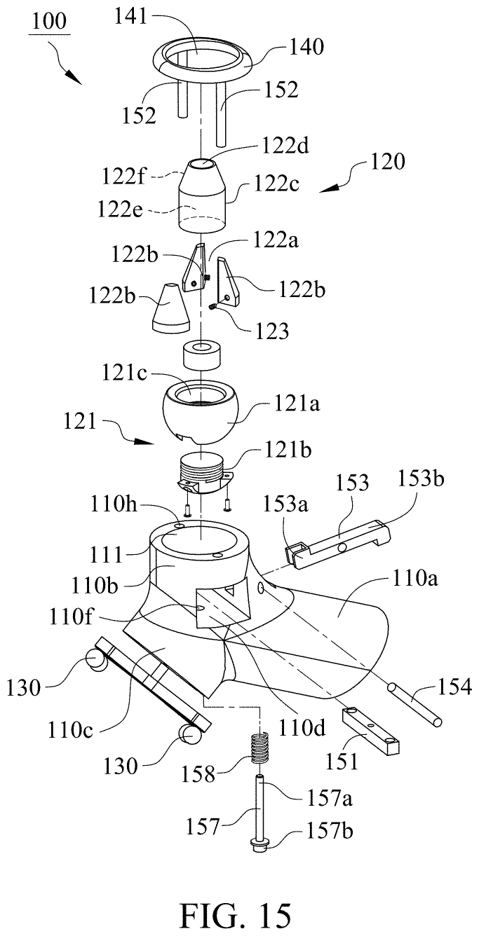

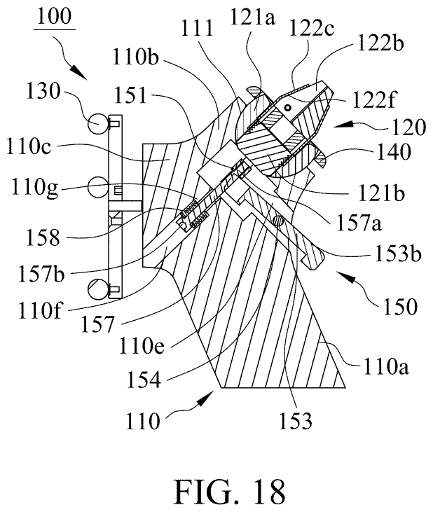

FIGS. 13 to 18 illustrate a calibration device 100 in accordance with a fourth embodiment of the present invention. The base 110 and the release mechanism 150 of the fourth embodiment are different to that of the second embodiment. With reference to FIGS. 15 and 17, in the fourth embodiment, the base 110 includes a restriction hole 110f communicating with the first space 110d, and the release mechanism 15 includes a linkage rod 157 and an elastic element 158. As shown in FIG. 17, there is a restriction bulge 110g in the restriction hole 110f, the linkage rod 157 of the release mechanism 150 is movably placed in the restriction hole 110f a first end 157a of the linkage rod 157 is coupled to the beam 151, a second end 157b of the linkage rod 157 is restricted in the restriction hole 110f and both ends of the elastic element 158 touch the second end 157b of the linkage rod 157 and the restriction bulge 110g, respectively.

With reference to FIG. 18, when pressing down the pressed end 153b, the pushing end 153a pushes the beam 151 to move the beam 151, the transmission rod 152, the restriction lid 140 and the linkage rod 157 together toward a first direction, as a result, the pivot portion 121 is not locked by the restriction lid 140 and the instrument holder 120 can be rotated or swung freely. Further, the elastic element 158 is compressed by the second end 157b of the linkage rod 157 to have a return force when the pressed end 153b is pressed down. With reference to FIG. 17, the compressed elastic element 158 is configured to push the second end 157b of the linkage rod 157 when no external force acts on the pressed end 153b of the lever 153 so that the beam 151, the transmission rod 152, the restriction lid 140 and the linkage rod 157 are moved together toward a second direction opposite to the first direction, and the restriction lid 140 is moved to contact the pivot portion 121 to prevent the instrument holder 120 from rotating.

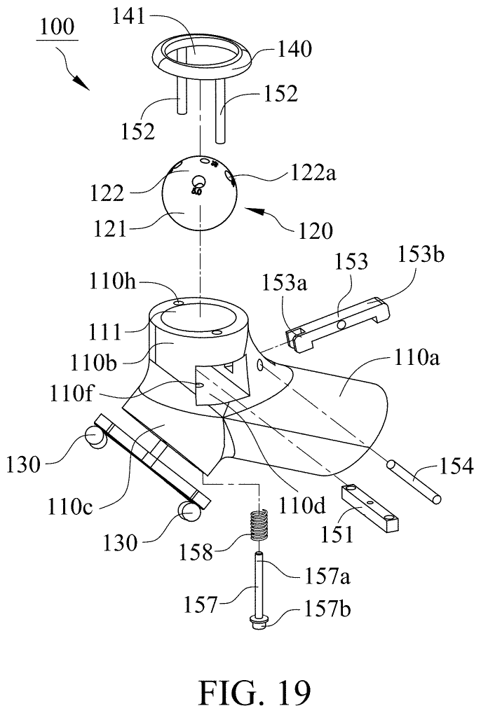

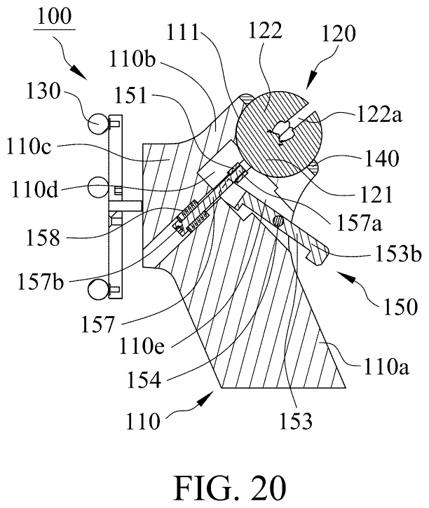

With reference to FIGS. 19 and 20, a calibration device 100 of a fifth embodiment of the present invention is disclosed. The difference between the fourth and fifth embodiments is that the instrument holder 120 of the fifth embodiment is a spheroid. In the fifth embodiment, the pivot portion 121 is also be blocked by or unblocked from the restriction lid 140 with the aid of the release mechanism 150.

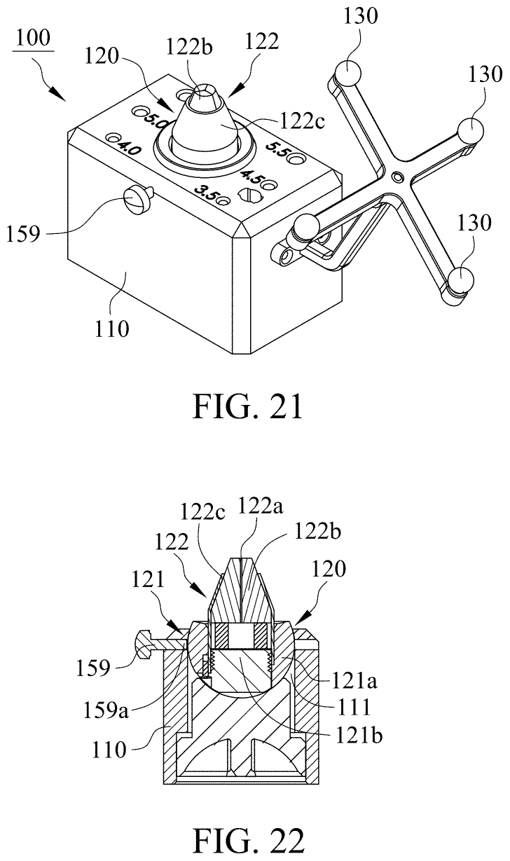

A calibration device 100 of a sixth embodiment of the present invention is disclosed in FIGS. 21 and 22. Different to the second embodiment, the release mechanism 150 of the sixth embodiment further includes a blocker 159 which is a bolt movably mounted on the base 110. A terminal 159a of the blocker 159 is used to contact the instrument holder 120 to prevent undesired rotation of the instrument holder 120.

With reference to FIG. 22, when the blocker 159 is screwed toward the instrument holder 120 to allow the terminal 159a to contact the instrument holder 120, the instrument holder 120 cannot be moved. Reversely, the instrument holder 120 is free to rotate or swing when the blocker 159 is screwed away from the instrument holder 120 to allow the terminal 159a not to contact the instrument holder 120.

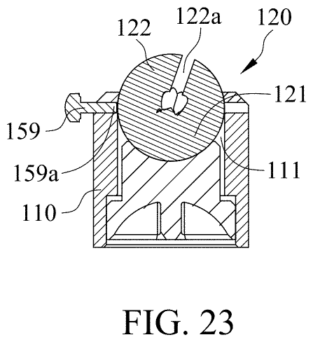

FIG. 23 shows a calibration device 100 of a seventh embodiment of the present invention. The difference between the sixth and seventh embodiments is that the instrument holder 120 is a spheroid in the seventh embodiment. The instrument holder 120 of the seventh embodiment is also prevented from undesired rotation by the blocker 159 and able to be rotated or swung freely when the blocker 159 is moved away from the instrument holder 120.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A calibration device, comprising:
a base;
an instrument holder pivotally mounted on the base, the instrument holder includes at least one insertion hole which is configured for insertion of a surgical instrument such that the surgical instrument is able to be selectively rotated or swung on the base; and
at least one reference frame marker mounted on the base.

2. The calibration device in accordance with claim 1, wherein the instrument holder includes a pivot portion and a insertion portion, the pivot portion is pivotally mounted in a first accommodation groove of the base, the insertion portion is mounted on the pivot portion, the at least one insertion hole is formed on the insertion portion and is revealed by the first accommodation groove.

3. The calibration device in accordance with claim 1, wherein the instrument holder is a spheroid, an axis passes through a center of the at least one insertion hole and a center of gravity of the instrument holder, and the center of gravity of the instrument holder is located on a hole bottom surface of the at least one insertion hole.

4. The calibration device in accordance with claim 1 further comprising a restriction lid, wherein the restriction lid includes a exposure hole communicating with the first accommodation groove and is configured to cover the pivot portion such that the pivot portion is caged between the first accommodation groove and the restriction lid, and the at least one insertion hole is exposed by the exposure hole.

5. The calibration device in accordance with claim 1, wherein the base includes a grip portion, a pivot portion and a marker mounting portion, the pivot portion is coupled to the grip portion and an included angle exists between the pivot portion and the grip portion, the first accommodation groove is recessed on the pivot portion, and the at least one reference frame marker is mounted on the marker mounting portion.

6. The calibration device in accordance with claim 1, wherein there are a plurality of insertion holes formed on the insertion portion, and each of the insertion holes has a diameter different to one another.

7. The calibration device in accordance with claim 1, wherein the insertion portion includes a plurality of grippers and a sleeve, the sleeve includes an opening, an accommodation space and a conical inner wall, the opening communicates with the accommodation space, the conical inner wall surrounds around the accommodation space, the pivot portion includes a body and a screw post, the screw post is located in a second accommodation groove of the body and is screwed with the sleeve, the grippers are mounted in the accommodation space and surround around the at least one insertion hole, the conical inner wall of the sleeve contacts the grippers, wherein the at least one insertion hole has a diameter that is adjustable by rotating the sleeve to change screwing position of the sleeve relative to the screw post.

8. The calibration device in accordance with claim 4 further comprising a release mechanism, wherein the release mechanism includes a beam, at least one transmission rod and a lever, the base includes a first space and a second space communicating with each other, the beam is mounted in the first space, both ends of the at least one transmission rod are coupled to the beam and the restriction lid respectively, the lever is pivotally mounted in the second space through a pivot and includes a pushing end and a pressed end, wherein when the pressed end is pressed, the pushing end is configured to push the beam such that the beam, the at least one transmission rod and the restriction lid are moved toward a first direction together to allow the restriction lid to release the pivot portion.

9. The calibration device in accordance with claim 8, wherein the release mechanism further includes at least one elastic element, both ends of the elastic element are fixed on the base and the beam respectively such that the beam, the at least one transmission rod and the restriction lid are able to be moved together toward a second direction opposite to the first direction to allow the restriction lid to contact the pivot portion.

10. The calibration device in accordance with claim 9, wherein the release mechanism further includes a positioning element mounted on the base, and the elastic element is fixed on the base by the positioning element.

11. The calibration device in accordance with claim 8, wherein the release mechanism further includes a linkage rod and an elastic element, the base includes a restriction hole communicating the first space, there is a restriction bulge in the restriction hole, the linkage rod is movably mounted in the restriction hole, a first end of the linkage rod is coupled to the beam and a second end of the linkage rod is restricted in the restriction hole, both ends of the elastic element contact the second end of the linkage rod and the restriction bulge respectively such that the beam, the at least one transmission rod, the restriction lid and the linkage rod are able to be moved together toward a second direction opposite to the first direction to allow the restriction lid to contact the pivot portion.

12. The calibration device in accordance with claim 1 further comprising a release mechanism, wherein the release mechanism includes a blocker movably mounted on the base, and a terminal of the blocker is configured to contact the instrument holder.
